(19) United States
(12) Patent Application Publication
(10) Pub. No.: US 2009/0124868 A1
(43) Pub. Date: May 14, 2009

(54) Lightweight portable trauma treatment and patient monitoring device

(76) Inventors: Peter Andrew Barnett, Costa Mesa, CA (US); Todd Douglas Kneale, Brea, CA (US); Steven Bruce Alexander, Rolling Hills Estates, CA (US); Terrance Paul Domae, Cerritos, CA (US); John Duval, Costa Mesa, CA (US)

Correspondence Address: STETINA BRUNDA GARRED & BRUCKER, 75 ENTERPRISE, SUITE 250, ALISO VIEJO, CA, 92656, US

(21) Appl. No.: 11/983,672

(22) Filed: Nov. 9, 2007

(51) Int. Cl. A61B 5/00

(52) U.S. Cl. 600/301

(57) ABSTRACT

There is provided a portable, lightweight, trauma treatment and patient monitoring device. The device comprises a base having a top surface and a pair of housings each having an inner surface. The housings are in pivotal communication with the base to enable selective articulation between a closed orientation and an open orientation. In the closed orientation, the inner surfaces of the housings are substantially parallel to the top surface of the base. In the open orientation, the pair of housings are in spaced opposed relation to each other and the inner surfaces are substantially perpendicular to the top surface of the base. The device also includes at least one bay located within at least one of the pair of housings. The bay is capable of receiving at least one medical monitoring/treatment device.

### CROSS-REFERENCE TO RELATED APPLICATIONS

Not Applicable.

### STATEMENT RE: FEDERALLY SPONSORED RESEARCH/DEVELOPMENT

Not Applicable

### BACKGROUND

The present invention relates in general to portable medical devices containing a plurality of medical care and monitoring devices. More particularly, the invention relates to a portable, lightweight, trauma treatment and patient monitoring device.

Sudden injury or disease may happen at a moment's notice. At the onset of such injury or disease, a patient often calls for the aid of an emergency response team. Upon arrival, members of the emergency response team assess the situation and diagnose the patient's condition. Oftentimes, patients cannot be sufficiently treated in the field, and require transport to a hospital or similar patient care center where more sophisticated equipment is readily available. Frequently, a patient's condition may worsen during the transportation period because the emergency response team does not have access to sufficient medical equipment in the field. In the case of serious injury or disease, it is common that the patient must be treated within an hour of the initial onset of the disease or injury to avoid even more serious injury, or possibly death. Treatment within the first hour, the so-called golden hour, increases the likelihood of survival and successful recovery. Many times, this time constraint cannot be met due to various reasons. For example, the patient may be in a remote location, more than an hour away from the closest patient care facility.

Although this problem occurs frequently in civilian situations, it may be magnified in battlefield settings, where significant injuries and disease commonly occur. Because of the degree of injury and disease encountered on the battlefield, the allowable timeframe for transporting the patient to a patient care center may be shortened. In addition, soldiers are likely located in remote, war-torn areas, which make rescue efforts very difficult. Many times, medical personnel are required to travel by foot to get to the patient's exact location. Under these circumstances, medical equipment must be carried to the patient by the emergency response personnel.

After arriving at the patient's location, emergency response personnel immediately begin diagnosing and treating the patient. Oftentimes, this involves the use of medical equipment, such as a ventilator. Such medical equipment is often placed beside the patient while the medical personnel tend to the patient. When the medical team determines that transport is necessary, the patient is placed on a stretcher or litter, and carried to the medical transport vehicle. At this time, any medical equipment attached to the patient may have to be disconnected or hand-carried by additional medical personnel. Disconnecting the instrumentality results in an undesirable disruption in the medical care and monitoring. Alternatively, hand-carrying the instrumentality requires extra personnel which may not be available.

Technological advances have provided devices allowing medical instrumentality to be stowed or carried along with the litter. Although such advances have greatly enhanced emergency care and response, current systems are large and require at least two medical care providers to transport the device to the patient's location. This requirement severely limits the range of medical care that may be provided to a patient. This is especially true in the case of natural disasters and battlefield environments where vehicular transport may not be a viable option. Although individual medical devices, such as defibrillators may be carried to the patient, current systems which integrate numerous medical devices are too large to be carried by one individual.

As such, there is a need in the art for a lightweight, man-portable trauma treatment and patient monitoring device.

### BRIEF SUMMARY

According to an aspect of the present invention, there is provided a man-portable, lightweight, trauma treatment and patient monitoring device. The device comprises a base having a top surface and a pair of housings each having an inner surface. Each housing includes an associated hinge pivotally connecting the housing to the base to enable selective articulation between a closed orientation and an open orientation. In the closed orientation, the inner surfaces of the housings are substantially parallel to the top surface of the base. In the open orientation, the pair of housings are in spaced opposed relation to each other and the inner surfaces are substantially perpendicular to the top surface of the base. The device also includes at least one bay located within at least one of the pair of housings. The bay is operative to receive at least one medical monitoring/treatment medical unit.

The present invention is a light weight, compact device capable of performing basic commonly accepted technological trauma treatment, patient physiological monitoring, and data logging and care for a critically injured or ill patient. The device is configured to be man-portable, requiring only one person to set up and operate. The present invention may be of particular value when pre-deployed to areas of expectant traumatic injury such as combat forward aid stations, medivac medical units, civil disaster relief caches, or austere environments without extensive rapid response capability such as rural or maritime search and rescue.

As was mentioned above, the device articulates between closed and open orientations. The open orientation may include an inboard position and an outboard position. The distance between the inner surfaces of the housings is smaller in the inboard position compared to the distance between the inner surfaces of the housings in the outboard position.

The trauma treatment and patient monitoring device may include additional attachments to facilitate transport. For instance, the device may include a mounting system including a wheelchair coupling members for engaging the wheelchair. In addition, a strap may be coupled to the base enabling a medical provider to carry the device. The device may also include a first attachment member coupled to the base permitting attachment to a litter. A second attachment member may also be coupled to the base to enable integration with an air casualty transport vehicle.

The device may further include an internal power source to enable operation of the device independent from an external power source. The internal power source may be disposed within at least one of the pair of housings and/or within the base.

At least one bay is located within the pair of housings. An embodiment of the present invention may include a bay located within each housing. In addition, the device may include at least two medical monitoring/treatment medical units within at least one bay. When at least two medical monitoring/treatment medical units are received within the device, the medical units may be in electrical communication with each other. The housings may be comprised of a housing body and a housing arm. The housing body may be pivotally connected to the housing arm. Furthermore, the housing arm may be pivotally connected to the base. In addition, a display device may be coupled to at least one of the housings. The display device is capable of displaying patient monitoring/treatment data.

The device may also include a data input coupled to at least one of the pair of housings. The data input enables a user to input data or commands to regulate operation of the medical monitoring/treatment medical units. In addition to a data input, the device may also include an input/output (I/O) port disposed on at least one of the housings. The I/O port is capable of connecting a sensor or treatment apparatus with the trauma treatment and patient monitoring device. Each I/O port is in electrical communication with at least one medical monitoring/treatment medical unit. The device may additionally include a transceiver operative to enable communication with a remote facility.

According to another embodiment of the present invention, there is provided a trauma treatment and patient monitoring device including a base having a top surface and a pair of housings. The pair of housings are in pivotal communication with the base, thereby enabling selective articulation between a closed orientation and a open orientation. In the closed orientation, the housings are substantially abutting each other, whereas in the open orientation, the housings are disposed on opposed sides of the base. The device also includes at least one bay located within at least one of the housings. The bay is capable of receiving at least one medical monitoring/treatment medical unit

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1A is a perspective view of the portable, lightweight, trauma treatment and patient monitoring device in an open orientation, specifically, the device is in an open orientation inboard position;

FIG. 1B is a perspective view of the portable, lightweight, trauma treatment and patient monitoring device in the open orientation, specifically, the device is in an open orientation outboard position;

FIG. 2 is a perspective view of the portable, lightweight, trauma treatment and patient monitoring device in a closed orientation;

FIG. 3A is a perspective view of the portable, lightweight, trauma treatment and patient monitoring device having a pair of housings in a closed orientation attached to a litter, the housings and litter shown in phantom, each housing containing four medical monitoring/treatment medical units;

FIG. 3B is a perspective view of the portable, lightweight, trauma treatment and patient monitoring device having a pair of housings in the open orientation attached to a litter, the housings and litter shown in phantom, each housing containing four medical monitoring/treatment medical units;

FIG. 4 is a perspective view of the portable, lightweight, trauma treatment and patient monitoring device including straps coupled to a base, the straps enabling a medical provider to carry the trauma treatment and patient monitoring device during transport;

FIG. 5 is a side view of the portable, lightweight, trauma treatment and patient monitoring device being carried on the back of the medical provider;

FIG. 6 is a perspective view of the portable, lightweight, trauma treatment and patient monitoring device in the open position, disposed on a litter;

FIG. 7 is a perspective view of the portable, lightweight, trauma treatment and patient monitoring device being attached to a litter;

FIG. 8 is a perspective view of the portable, lightweight, trauma treatment and patient monitoring device coupled to a wheelchair; and

FIG. 9 shows the portable, lightweight, trauma treatment and patient monitoring device disposed on a litter, wherein a patient is lying on the litter, and the litter is being carried by two emergency medical personnel.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

The present invention is a lightweight, compact system of integrated medical, data and communication systems packaged to facilitate and support basic commonly accepted technological trauma treatment and care for a critically injured patient. This package is comprised of a durable housing containing an assortment of subsystem medical units representing a basic set of diagnostic, therapeutic and data management functionalities as required for at least echelon one patient resuscitation and care. The medical units are removable as individual medical units to enable maintenance or system reconfiguration in response to a patient's specific requirements. The present invention may be of particular value when pre-deployed in areas of expectant traumatic injury such as combat forward aid stations, medivac medical units, civil disaster relief caches, or austere environments without extensive rapid response capability such as rural or maritime search and rescue. The aim of the device is to shorten the time period between the time of injury and the delivery of ICU level medical functionality to as early in the first hour is possible.

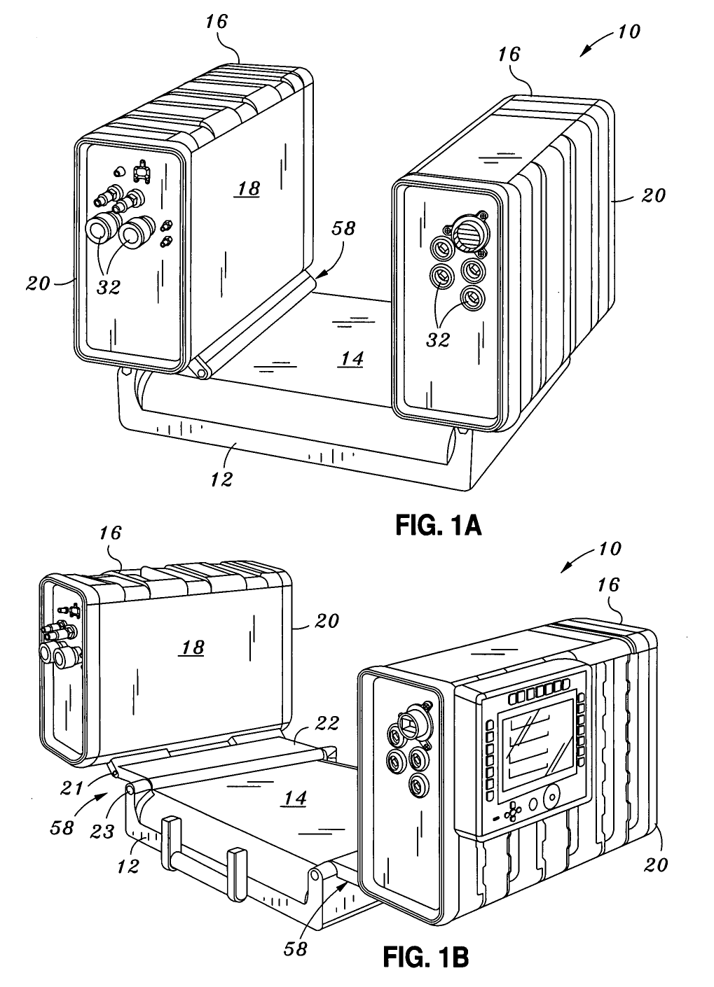

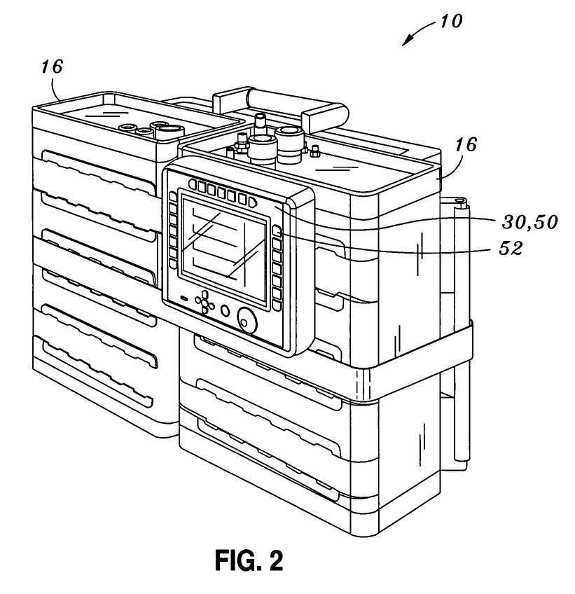

Turning now to FIGS. 1A-1B, which depict an embodiment of the present invention, there is provided a portable, lightweight, trauma treatment and patient monitoring device 10. The device 10 comprises a base 12 having a top surface 14 and a pair of housings 16. Each housing 16 includes an inner surface 18. The housings 16 are in pivotal communication with the base 12 to enable selective articulation between a closed orientation and an open orientation. FIGS. 1A-1B shows an embodiment in the open orientation, wherein the pair of housings 16 are in spaced opposed relation to each other and the inner surfaces 18 are substantially perpendicular to the top surface 14 of the base 12. Preferably, the open orientation includes an inboard position, as shown in FIG. 1A, and an outboard position, as shown in FIG. 1B. The device 10 may include a translational assembly 58 to enable movement between the inboard and outboard positions. The translational assembly 58 may include a pivot arm 22, and first and second hinges 21, 23. The first hinge 21 pivotally connects the pivot arm 22 to the housing body 20. The second hinge 23 pivotally connects the pivot arm 22 to the base 12. In both the inboard and outboard positions, the inner surfaces 18 of the housings 16 are substantially parallel to the top surface 14 of the base 12; however, the distance between the inner surfaces 18 varies between the inboard and outboard positions. In particular, the distance between the inner surfaces 18 of the housings 16 is smaller in the inboard position than the distance between the inner surfaces of the housings 16 in the outboard position.

FIG. 2 shows the device 10 in the closed orientation, wherein the inner surfaces 18 of the housings 16 are substantially parallel to the top surface 14 of the base 12. Although the embodiment shown in FIG. 2 shows the inner surfaces 18 of the housings 16 substantially abutting the top surface 14 of the base 12, it is understood that other embodiments may include inner surfaces 18 that do not abut the top surface of the base, yet are nonetheless parallel to the top surface. Preferably, the device 10 remains fully operational in both the closed and open orientations. The fact that the device 10 is in the closed orientation does not necessarily imply that the device 10 is not operational. The articulations between the closed and open orientations are only required to adapt the device 10 to a stretcher, litter, or patient; they are not necessarily articulations between an operational and non-operational position.

According to another embodiment of the invention, the housings 16 may be configured such that the inner surface 18 of each housing 16 is not substantially parallel to the base 12 when in the closed orientation. In such an embodiment, the closed orientation is defined as the position wherein the housings 16 are substantially abutting each other. In the corresponding open orientation, the housings 16 are disposed on opposed sides of the base 12. An example of such an embodiment includes a device 10 comprising housings 16 having a triangular cross section.

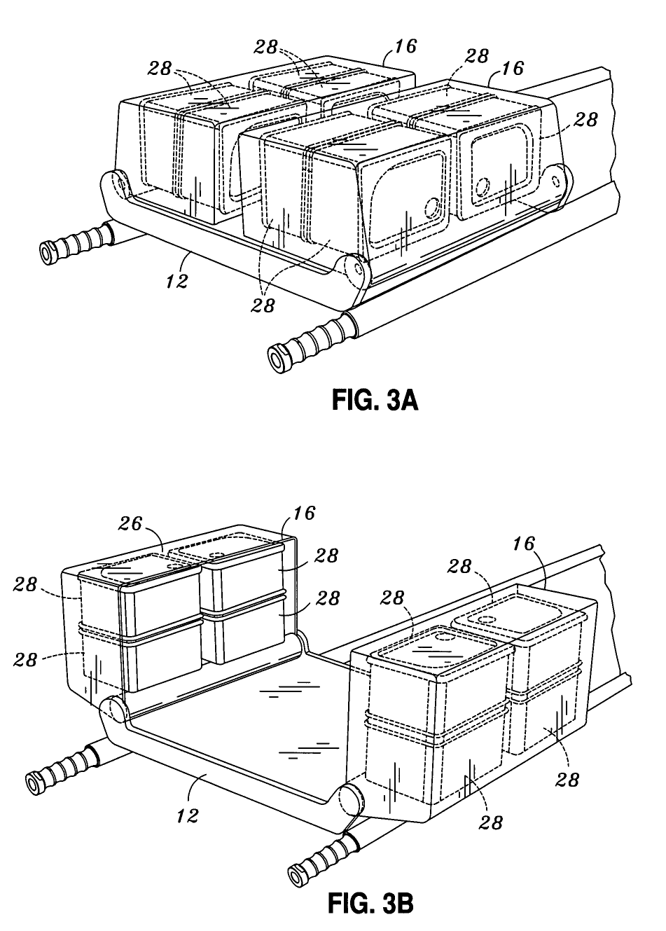

It is contemplated that the device 10 also includes at least one bay 26 located within at least one of the pair of housings 16. FIGS. 3A and 3B are perspective views of the device 10, wherein the pair of housings 16 are shown in phantom. FIG. 3A depicts the device 10 in the closed orientation, whereas FIG. 3B shows the device 10 in the open orientation. Located within at least one of the housings 16 is at least one bay 26. The bay 26 is the area within the housing 16 wherein at least one medical monitoring/treatment medical unit 28 is received. According to various embodiments of the present invention, the bay 26 may be designed to receive only one medical unit 28, or the bay 26 may be designed to receive multiple medical units 28. For instance, in one embodiment, there may be one housing 16 having a bay 26 designed to receive only one medical unit 28, while the other housing 16 may include a bay 26 designed to receive multiple medical units 28. When at least two medical units 28 are received within the bay 26, the medical units 28 may be in electrical communication with each other. As such, the medical units 28 may share power or data in order to facilitate patient treatment or monitoring. For instance, a patient's age, weight, sex, etc. may be entered once and communicated between the medical units 28 rather than having to enter the same information for each medical unit 28. In the embodiment shown in FIGS. 3A and 3B, both housings 16 include a bay 26, wherein each bay 26 includes four medical units 28.

As discussed above, the bay 26 is capable of receiving at least one medical monitoring/treatment medical unit 28. As used herein, a medical monitoring/treatment medical unit 28 is a compact medical unit, which houses hardware operative to regulate medical functions, including patient treatment and/or monitoring functions. Exemplary medical functions capable of being performed by the medical units 28 include, but are not limited to a clinical analyzer, a defibrillator, infusion pumps, suction/aspiration, ventilation, CO₂/O₂ flow, oxygen generator, and physical monitoring including pulse oximetry, temperature monitoring, respiratory rate/cardiac output monitoring, invasive and non-invasive blood pressure monitoring, ECG, ventilating and defibrillating. Medical units 28 may be swapped into and out of the bay 26 as needed. For instance, if a defibrillator and heart rate monitor are needed, those medical units 28 are placed within the bay 26. Other medical units 28 may be removed from the bay 26 if room is needed for higher priority medical units 28. It is contemplated that the medical units 28 may be hot-swappable during operation of the device 10. That is to say that medical units 28 may be added or removed as necessary without turning the whole system off. This capability facilitates bringing additional functionality online as needed.

According to various embodiments, the housing 16 may include a housing body 20 and housing arm 22. The housing body 20 may be pivotally connected to the housing arm 22. In this regard, a first hinge 21 may pivotally couple the housing body 20 to the housing arm 22. Likewise, the housing arm 22 may be pivotally connected to the base 12. A second hinge 23 may pivotally couple the housing arm 22 to the base 12.

It may also be desirable to enable the housing 16 to translate between the inboard and outboard positions in a plane that is substantially parallel to the base 12. Such translation enables the distance between the housings 16 to vary when the housings 16 are in the open orientation. It may be desirable to shorten the distance between the housings 16 to allow the device 10 to become more compact. For instance, the patient may be located in an area with a limited amount of space to deploy the device 10. As such, the housings 16 may translate to decrease the distance between the housings 16. In other circumstances, it may be advantageous to increase the distance between the housings 16 to create may room to work on the patient, if space allows. Therefore, the housing 16 may be in translatable communication with the base 12. In one embodiment of the invention, the housing 16 may be translatably coupled to the base 12. In another embodiment, the housing body 20 may be translatably coupled to the housing arm 22. In such an embodiment, the housing body 20 may be able to translate in a plane that is substantially parallel to the housing arm 22. In a further embodiment, the housing arm 22 may be translatably coupled to the base 12, thereby allowing the housing arm 22 to translate in a plane that is substantially parallel to the base 12.

According to another embodiment of the invention, the device 10 may include a base 12 having a variable length, wherein the length is defined as the distance between the points at which each housing 16 is attached to the base 12. Such a base 12 includes a first base portion and a second base portion, wherein the first and second base portions are in translatable communication with each other.

As was described above, each medical unit 28 is capable of regulating medical treatments and/or monitoring functions. Many treatment/monitoring functions require the use of sensor or treatment apparatus 48. As used herein, a sensor or treatment apparatus 48 is medical equipment interfacing directly with the patient. Examples of sensor or treatment apparatus 48 may include, but are not limited to defibrillator paddles or a ventilator circuit. Preferably, the device 10 comprises a treatment assembly 31 including an input/output (I/O) port 32 and a sensor/treatment apparatus 48. In one embodiment of the invention, the device 10 may include at least one I/O port 32 disposed on at least one of the housings 16. Although one embodiment of the invention may only include one I/O port 32 on one housing 16, it is understood that other embodiments may include a plurality of I/O ports 32 disposed on each housing 16. The I/O port 32 enables electrical communication between the sensor or treatment apparatus 48 and its corresponding medical monitoring/treatment medical unit 28. As such, the medical unit 28 is able to control the sensor or treatment apparatus 48. In addition, the sensor/treatment apparatus 48 is able to transmit signals to the medical units 28 via the I/O ports 32. For instance, a medical unit 28 for monitoring a heart rate may send signals to heart rate monitoring apparatus via the I/O port 32. As the heart rate monitoring apparatus receives data relating to the patient's heart rate, it may send that data to the medical unit 28 via the I/O port 32.

In another embodiment of the invention, a display device 30 may be coupled to at least one of the housings 16. The display device 30 is capable of displaying patient monitoring and/or treatment data. Patient monitoring and/or treatment data may include any data relating to the patient and his current condition. Such data may be produced by any of the medical units 28 and may include, but is not limited to a patient's heart rate, blood pressure, body temperature, ECG reading, etc. The display device 30 may be coupled to only one housing 16, or each housing 16 may include at least one display device 30. The display device 30 may also be an external device that is capable of connecting with one of or both of the housings 16 through an I/O port 32.

In still another embodiment of the present invention, a data input 50 may be coupled to at least one housing 16. The data input 50 preferably includes a user interface 60 having an input member 52 to enable a user to input data/commands. The data input 50 enables a user to input data and/or commands to regulate operation of at least one medical unit 28. In other words, the data input 50 enables a medical provider to communicate parameters, patient conditions, modes, etc., to the medical units 28. For example, the data input 50 may be used to input the height, weight, age, sex of the patient, and/or other data relating to the patient, the patient's condition, the medical provider, etc. When multiple medical units 28 are received within the bay 26, the data input 50 may allow a user to regulate operation of all medical units 28 located within the device 10. In one embodiment of the invention, the data input 50 may be a touch screen monitor, allowing the operator to input information by touching the screen. In another embodiment, the data input 50 and the display device 30 may be integrated into one piece of hardware being capable of displaying data, and also enabling an operator to input data.

In operation, it is expressly contemplated that a portion of the patient's body may be placed on the device 10. It is understood that upon arriving at the patient's location, the device 10 may be positioned in the open orientation. Once in the open orientation, a patient's legs may rest on the top surface 14 of the base 12, between the two housings 16, as is shown in FIG. 9. As such, the patient is in close proximity to the device 10, and is also in a good position to receive medical treatment and care. However, if the patient's legs cannot be placed on the top surface 14 of the base 12, it is understood that the device 10 may be placed in close proximity to the patient so as to enable medical treatment and care.

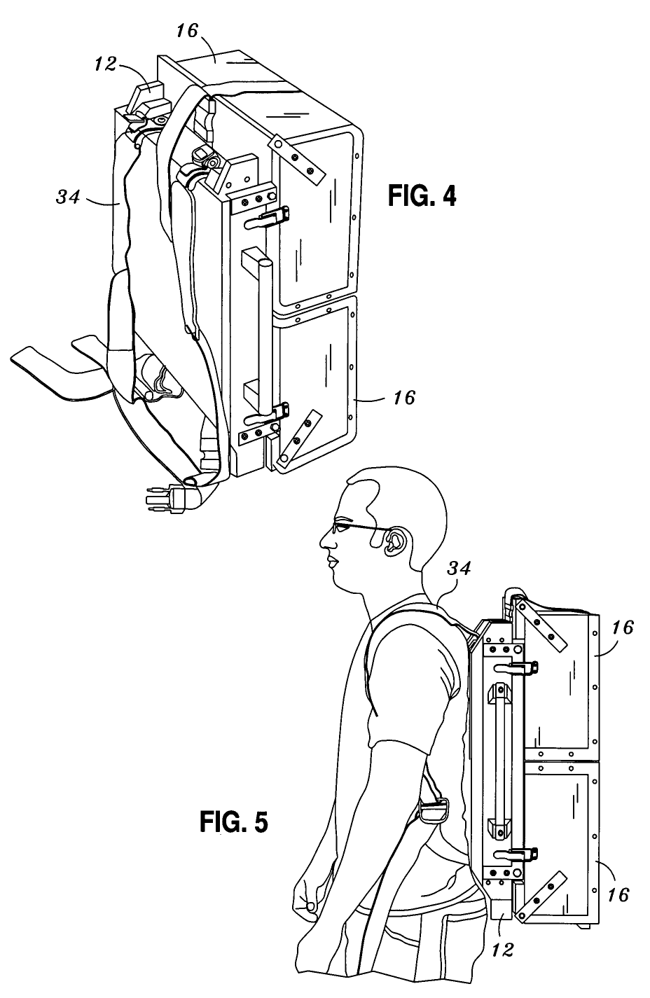

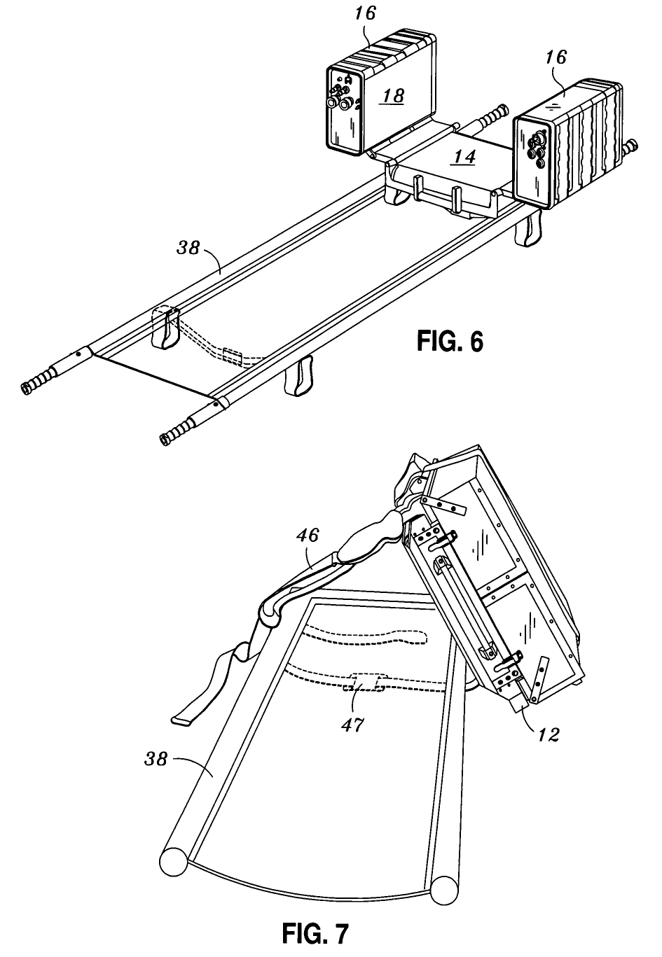

Turning now to FIGS. 4 and 5, it is contemplated that the present invention is a highly portable trauma treatment and patient monitoring device 10 that may be adapted for different uses or different environments. The device 10 has an ideal weight of less than thirty pounds and could be carried to a patient's location and open by one person. In order to increase the portability of the device 10, additional attachments may be coupled to the device 10 to facilitate different transport environments. In one embodiment, the device 10 may include at least one shoulder strap 34 attached to the base 12 to enable a medical provider to carry the device 10. As shown in FIG. 4, the device 10 includes two straps 34 coupled to the base 12. The straps 34 allow a medical provider to wear the device 10 over his shoulder or back, much like a backpack or satchel. FIG. 5 shows the device 10 in the closed orientation, wherein the device 10 is being carried by a medical provider. The strap(s) 34 enable a medical provider to transport the device 10 into hard to reach locations. This may be particularly useful in natural disaster areas or battlefield environments where transport by vehicle may not be an option.

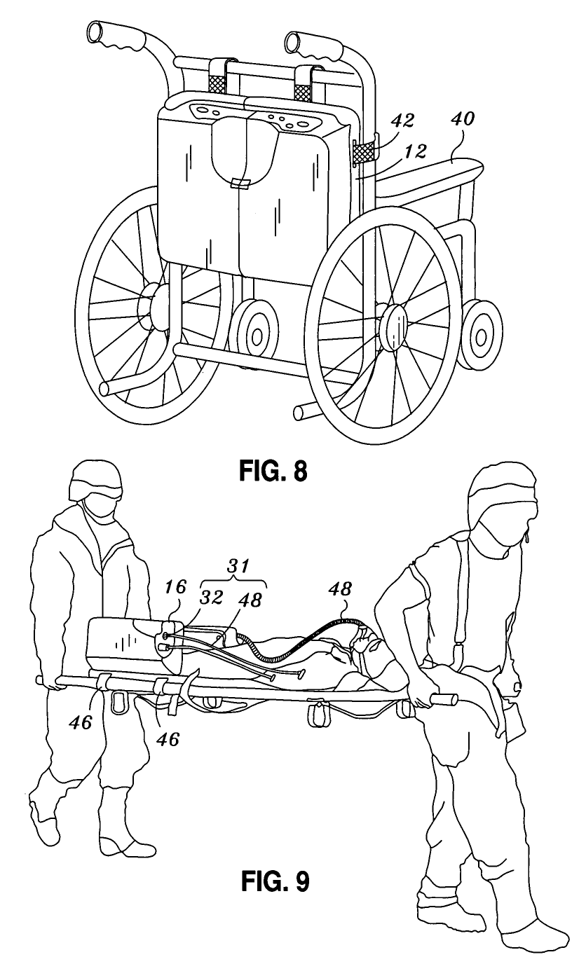

According to other embodiments, the present invention may include additional attachments to facilitate different transport environments. FIG. 8 shows a device 10 in the closed orientation attached to the back of a wheelchair 40. The device 10 may include a mounting system 42 coupled to the base 12, capable of attaching to a wheelchair 40. Attaching the device 10 to a wheelchair 40 may be useful to prevent the interruption of treatment or monitoring. For instance, if a wheelchair 40 becomes available, it may be desirable to continue treatment and monitoring while the patient sits in the wheelchair 40. Rather than disconnecting the equipment, or having someone carry the device 10 alongside the wheelchair 40, the mounting strap 42 enables the device 10 to be attached to the wheelchair 40. In one embodiment of the invention, the mounting strap 42 may include hooks that enable the device 10 to be clipped to or hung from the frame of the wheelchair 40.

In addition to attaching to a wheelchair 40, the device 10 may also be attachable to a litter 38, as is shown in FIG. 7. The device 10 may include a first attachment assembly 46 including a litter coupling member 47. The treatment assembly is coupled to the base 12 and the litter coupling member permits attachment to the litter 38. In one embodiment, the attachment assembly 46 may be a hook coupled to the base 12, wherein the hook clips to the litter 38 for securement. The device 10 may also be designed so that the base 12 fits within the width of a litter 38. Standard, industry-wide dimensions may be used to determine the width. For example, a standard NATO litter may be used 38. In addition, as was described above, one embodiment of the invention includes a base 12 having a variable length. A variable base 12 may be beneficial when trying to couple the device 10 to a litter. A user may place the device 10 within the litter and adjust the length of the base 12 such that the base 12 fits within the litter.

In another embodiment, the device 10 may include other attachment members enabling integration with other transportation vehicles. The device 10 may integrate with a medical transport vehicle, such as an ambulance, or air casualty transport vehicle. Such integration may stabilize the device 10 and patient during transport. For instance, if the patient is being transported to a hospital or other medical care center via air transport, the integration may provide stability during aggressive flying maneuvers, or during turbulence.

It is contemplated that the device 10 may receive power from both internal and external power sources. The external source may be used to both power the device 10 and recharge the interior power source 44. The device 10 may operate exclusively on an internal power source 44 if an external power source is not available. According to one embodiment, the device 10 may comprise an internal power source 44 disposed within at least one of the pair of housings 16. As such, the device 10 may include an internal power source 44 in one housing 16, or in both housings 16. In another embodiment, the internal power source 44 may also be disposed within the base 12. The power source, whether internal or external, is in electrical communication with each medical unit 28, and thereby provides power to each medical unit 28. According to an embodiment of the invention, the internal power source 44 may be comprised of at least two batteries. The batteries may be hot-swappable, meaning that one of the batteries may be removed and replaced at any time while the system is functioning. This assures continuous operation by allowing freshly recharged batteries to be brought on-line at any time.

According to another embodiment of the present invention, the device 10 may additionally include a transceiver operative to enable communication with a remote facility. The transceiver is capable of transmitting data to and receiving data from a remote facility. The transceiver may use wireless technology such as WiFi, Bluetooth, or other wireless technology known or later developed, to communicate with the remote facility. The remote facility may be a hospital or other medical care center. The transceiver may be used to alert the medical facility that the medical team is traveling to their facility. In addition, the patient's vital signs and/or other conditions may also be communicated via the transceiver. The transceiver may also be used to transmit a patient's medical file/history to the medical team, as well as to transmit diagnosis or treatment information that may be useful to the medical team.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A portable, lightweight, trauma treatment and patient monitoring device comprising:
a base having a top surface;
a pair of housings each having an inner surface, each housing having an associated hinge pivotally connecting the housing to the base to enable selective articulation between a closed orientation wherein the inner surfaces of the housings are substantially parallel to the base top surface, and an open orientation wherein the pair of housings are in spaced opposed relation to each other and the inner surfaces are substantially perpendicular to the base top surface; and
at least one bay located within at least one of the pair of housings, the bay being capable of receiving at least one medical monitoring/treatment medical unit.

2. The trauma treatment and patient monitoring device of claim 1, wherein the housing includes a housing body and a housing arm, the housing arm being pivotally coupled to the housing body.

3. The trauma treatment and patient monitoring device of claim 2, further comprising a translational assembly including first and second hinges, the first hinge pivotally connecting the housing arm to the housing body, the second hinge pivotally connecting the housing arm to the base, wherein the translational assembly is operative to allow movement between an open orientation inboard position and an open orientation outboard position, wherein the distance between the inner surfaces of the housings is smaller in the inboard position compared to the distance between the inner surfaces of the housings in the outboard position.

4. The trauma treatment and patient monitoring device of claim 1, further comprising a mounting system coupled to the base, the mounting system including wheelchair coupling members for engaging a wheelchair.

5. The trauma treatment and patient monitoring device of claim 1, further comprising at least one shoulder strap coupled to the base enabling a medical provider to carry the trauma treatment and patient monitoring device during transport.

6. The trauma treatment and patient monitoring device of claim 1, further comprising an internal power source disposed within at least one of the pair of housings.

7. The trauma treatment and patient monitoring device of claim 1, further comprising an internal power source disposed within base.

8. The trauma treatment and patient monitoring device of claim 1, further comprising a litter coupling member coupled to the base, the litter coupling member being configured to engage the litter.

9. The trauma treatment and patient monitoring device of claim 1, wherein at least two medical monitoring/treatment medical units are disposed within the at least one bay.

10. The trauma treatment and patient monitoring device of claim 9, wherein the at least two medical monitoring and treatment medical units are in electrical communication with each other.

11. The trauma treatment and patient monitoring device of claim 1, further comprising a display device coupled to at least one of the pair of housings, the display being operative to display patient monitoring/treatment data.

12. The trauma treatment and patient monitoring device of claim 1, further comprising a data input coupled to at least one of the pair of housings, the data input having a user interface including an input member for enabling a user to input data/commands to regulate operation of the at least one medical monitoring/treatment medical unit.

13. The trauma treatment and patient monitoring device of claim 12, wherein the input member is a touch-screen.

14. The trauma treatment and patient monitoring device of claim 1, further comprising at least one treatment assembly including an I/O port disposed on at least one of the pair of housings, and a sensor/treatment apparatus, the at least one I/O port operative to couple the sensor/treatment apparatus with the trauma treatment and patient monitoring device, the at least one treatment assembly being in electrical communication with the at least one medical monitoring/treatment device.

15. The trauma treatment and patient monitoring device of claim 14, wherein the sensor/treatment apparatus is a defibrillator paddle.

16. The trauma treatment and patient monitoring device of claim 14, wherein the sensor/treatment apparatus is a ventilator circuit.

17. The trauma treatment and patient monitoring device of claim 1, further comprising a transceiver in electrical communication with the at least one medical monitoring/treatment medical unit, the transceiver being operative to enable communication of medical monitoring/treatment data to a remote facility.

18. A portable, lightweight, trauma treatment and patient monitoring device comprising:
a base having a top surface;
a pair of housings, each housing having as associated hinge connecting the housing to the base to enable selective articulation of the housing between a closed orientation wherein the housings are substantially abutting each other in a horizontal orientation, and a open orientation, wherein the housings are disposed on opposing sides of the base in a vertical orientation;
at least one bay located within at least one of the pair of housings, the bay being capable of receiving at least one medical monitoring/treatment medical unit.

19. The trauma treatment and patient monitoring device of claim 18, wherein each housing includes a plurality of medical monitoring/treatment medical units.

20. The trauma treatment and patient monitoring device of claim 18, further comprising a display device coupled to the at least one monitoring/treatment medical unit, the display being capable of displaying patient monitoring/treatment data.

21. The trauma treatment and patient monitoring device of claim 18 further comprising a data input coupled to at least one of the pair of housings and in electrical communication with the at least one medical monitoring/treatment medical unit, the data input enabling a user to input data/commands to regulate operation of the at least one medical monitoring/treatment medical unit.

22. The trauma treatment and patient monitoring device of claim 18 further comprising at least one I/O port disposed on at least one of the pair of housings, the at least one I/O port being capable of connecting a sensor/treatment apparatus with the trauma treatment and patient monitoring medical unit, the at least one I/O port being in electrical communication with the at least one medical monitoring/treatment medical unit.

23. The trauma treatment and patient monitoring device of claim 18, wherein the open orientation includes an inboard position and an outboard position, wherein the distance between the housings is smaller in the inboard position compared to the distance between the housings in the outboard position.

24. The trauma treatment and patient monitoring device of claim 18, further comprising an internal power source disposed within base.

25. A patient monitoring treatment platform comprising:
a litter; and
a trauma treatment and patient monitoring device comprising:
an attachment assembly including a litter coupling member operative to detachably couple the trauma treatment and patient monitoring device to the litter;
a base having a top surface;
a pair of housings each having an inner surface, each housing having an associated hinge pivotally connecting the housing to the base to enable selective articulation between a closed orientation wherein the inner surfaces of the housings are substantially parallel to the base top surface, and an open orientation wherein the pair of housings are in spaced opposed relation to each other and the inner surfaces are substantially perpendicular to the base top surface; and
at least one bay located within at least one of the pair of housings, the bay being capable of receiving at least one medical monitoring/treatment medical unit.
